SEQUENCE LISTING (2 sequences)

SEQ ID NO: 1 - protein, 15 aa, Artificial
  Synthetic peptide
CYDDLGKGGNEESTK

SEQ ID NO: 2 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

(19) United States
(12) Patent Application Publication
(10) Pub. No.: US 2007/0202537 A1
(43) Pub. Date: Aug. 30, 2007

(54) BIOMARKERS FOR ALS

(75) Inventors: Vishwanath Lingappa, San Francisco, CA (US); Arie Lev Gurzman, Lafayette, CA (US); Jian Liu, San Francisco, CA (US)

Correspondence Address: FOLEY AND LARDNER LLP;SUITE 500, 3000 K STREET NW, WASHINGTON, DC, 20007, US

(73) Assignee: Prosetta Corporation

(21) Appl. No.: 11/567,142

(22) Filed: Dec. 5, 2006

Related U.S. Application Data
(60) Provisional application No. 60/742,726, filed on Dec. 5, 2005.

(51) Int. Cl. G01N 33/53; C07K 16/40; C07K 7/08

(52) U.S. Cl. 435/7.1; 530/388.26; 530/327

(57) ABSTRACT

Provided are methods for diagnosing sporadic and familial forms of amyotrophic lateral sclerosis (“ALS”) that detect conformers or conformer patterns of the copper-zinc superoxide dismutase-1 (“SOD-1”) enzyme that are common to sporadic or familial ALS individuals but distinct from SOD-1 conformers of normal individuals. Methods of identifying candidate drugs that modulate SOD-1 conformer formation also are provided.

### CROSS-REFERENCE TO RELATED APPLICATIONS

This application claims priority under 35 U.S.C. § 119(e) to U.S. Provisional Application U.S. Ser. No. 60/742,726, filed Dec. 5, 2005, which is incorporated by reference in its entirety into the present disclosure.

### 1 BACKGROUND OF THE INVENTION

1.1 Field of the Invention

The present invention relates generally to the field of human amyotrophic lateral sclerosis (“ALS”) and to methods of diagnosing or predicting ALS and identifying potential ALS therapeutic agents. The invention has applications in the fields of: diagnostics, medicinal chemistry, and neurological medicine.

1.2 The Related Art

Amyotrophic lateral sclerosis (“ALS”), also called Lou Gehrig's disease after the famous baseball player who died from the disease, is a progressive fatal neurological affliction that affects as many as 40,000 Americans, with 5,000 new cases occurring in the United States each year. ALS is characterized by the gradual steady degeneration of certain nerve cells in the brain cortex, brain stem and spinal cord involved in voluntary movement. The result of the degeneration is complete paralysis and death.

ALS manifests itself in different ways, depending on which muscles weaken first. Symptoms may include tripping and falling, loss of motor control in hands and arms, difficulty speaking, swallowing or breathing, persistent fatigue, and twitching and cramping (sometimes quite severely). ALS often strikes in mid-life and is usually fatal within five years after diagnosis.

Like many other neurodegenerative diseases, only a small percentage (about 10%-15%) of ALS is inherited. Genetic epidemiology of ALS has revealed at least six chromosome locations accountable for the inheritance of disease (ALS1 to ALS6, reviewed by Majoor-Krakauer et al., 2003). Among these, three genes have been identified. The first was identified in 1993 as the cytosolic Cu/Zn superoxide dismutase (SOD-1) gene that accounts for 20% of the autosomal dominant form of ALS (Rosen et al., 1993). The second was named as Alsin, a potential guanine-nucleotide exchange factor (GEF) responsible for the juvenile recessive form of ALS (Hadano et al., 2001; Yang et al., 2001). The third is ALS4 that encodes for a DNA/RNA helicase domain containing protein called Senataxin identified to be linked to the autosomal dominant form of juvenile ALS (Chen et al., 2004). Most recently, a mutation in the vesicle associated membrane protein/synaptobrevin associated membrane protein B (VAPB) in a new locus called ALS8, was reported to be associated with an atypical form of ALS (Nishimura et al., 2004).

Looking for biomarkers for ALS remains a strong interest for physicians, patients, as well as researchers. Since more than 90% of ALS manifests in a sporadic fashion, there is no convenient existing markers (genetic or biochemical) that one can use to diagnose and/or assess disease progression of ALS (Rachakonda et al., 2004; Malaspina and de Belleroche, 2004; Gooch et al., 2004; Kalra et al., 2004; Simpson et al., 2004). Indeed, InnoCentive, a forum for encouraging research on specific projects by providing financial prises, announced on 13 Nov. 2006 that Prize4Life, Inc., a non-profit organization founded to accelerate research in Lou Gehrig's disease, had offered a one million dollar incentive for the identification of an ALS biomarker, since, according to Robert H. Brown, M. D., D. Phil, a member of the Prize4Life Scientific Board: “[v]alid biomarkers [for ALS] will enhance our understanding of the pathological process in ALS and our ability to gauge treatment efficacy.” Currently, diagnosis of ALS is made from combination of clinical and neurophysiological assessment. While common symptoms are observed in both familial and sporadic ALS (FALS and SALS) patients, variations in a number of disease aspects including the site of onset, disease manifestation, and progression exist among individuals (Strong and Rosenfeld, 2003).

Differences in protein structures resulting from mutations in the SOD-1 gene have long been viewed as the original sources of gained toxic properties that cause motor neuron death in mutant SOD-1-mediated FALS models. Altered SOD-1 protein conformation can be detected by X-Ray crystallography and solution NMR. Some mutant SOD-1s also displayed higher propensity to form aggregates more strongly than the wild type protein (Ross and Poirier, 2004). However, a direct correlation between different SOD-1 conformers and FALS and SALS has not been demonstrated heretofore.

If reliable biomarkers are available then not only can the physicians objectively diagnose ALS, but they may also be able to assess the rate of disease progression. More importantly, biomarkers will be extremely useful to evaluate efficacy of therapeutic drug testing for the treatment of ALS. Identification of biomarkers is equally important for basic research in ALS. Studies in both human ALS patients and rodent models of ALS based on mutations in the SOD-1 for the past decade have clearly demonstrated that motor neurons die via mitochondria-mediated apoptotic pathways (reviewed by Przedborski, 2004). In addition, motor neurons do not die alone; they are inevitably influenced by other cells in the surroundings (Clement et al., 2003). In other words, there can be a global disturbance in cellular conditions in the course of developing ALS.

Thus, providing reliable biomarkers that correlate with a cellular signature of pathological events in ALS will contribute greatly to our understanding of disease mechanisms. The present invention meets these and other needs.

### 2 SUMMARY OF THE INVENTION

The present invention provides biomarkers for ALS and methods of diagnosing patients who may have ALS. In a first aspect, the present invention provides a method of diagnosing ALS, comprising detecting an SOD-1 biomarker correlated with the presence of ALS in a patient. In one embodiment, the method of the invention further includes isolating a sample from the peripheral tissue of the patient. In a more specific embodiment, the isolating includes collecting a sample from the peripheral muscle, liver, or spinal fluid of the patient; in still more specific embodiments, the SOD-1 biomarker is biotinylated.

In still another aspect, the present invention provides an antibody having substantially specific affinity to an SOD-1 conformer that is associated with the presence of ALS in a patient. In one embodiment, the conformer is associated with the presence of SALS. In another embodiment, the conformer is associated with the presence of FALS.

In yet another aspect, the present invention provides a polypeptide having the sequence: CYDDLGKGGNEESTK (SEQ ID NO: 1), which can be used to raise SOD-1 antibodies as described herein.

In still another aspect, the present invention provides a method of diagnosing whether an individual has sporadic or familial ALS, the method comprising: obtaining a cell free extract derived from cells or tissue taken from an individual suspected of having sporadic or familial ALS; and identifying one or more SOD-1 conformer(s) in the cell free extract by one or more physical characteristics common to sporadic or familial ALS but distinctive from that of normal individuals. In some embodiments, such characteristics are selected from the group consisting of: immunological detection, electrophoretic mobility, and sedimentation rate. In other embodiments, the characteristics include differential reactivity to chemical reagents. In some embodiments of this method, the immunological detection is determined using SOD-1 conformer-specific monoclonal or polyclonal antibodies. In still more specific embodiments, the conformer-specific monoclonal or polyclonal antibodies are characterized by differential reactivity with SOD-1 prepared by in vitro synthesis of wild-type mRNA versus SOD-1 obtained by in vitro synthesis of mutant mRNA. In still more specific embodiments, the conformer-specific monoclonal or polyclonal antibodies are further characterized by a substantial reduction or complete loss of differential reactivity when immunological detection is evaluated using in vitro synthesized SOD-1 that has been denatured prior to antibody binding. In yet more specific embodiments, the conformer-specific monoclonal or polyclonal antibodies are characterized by differential reactivity shown by binding with mutant in vitro synthesized SOD-1 but not wild-type in vitro synthesized SOD-1, or vice versa. In some embodiments, the conformer-specific monoclonal or polyclonal antibodies are further characterized by a substantial reduction or complete loss of differential reactivity when immunological detection is evaluated using in vitro synthesized SOD-1 that has been denatured prior to antibody binding. In still other embodiments, the immunological detection comprises immunoprecipitation of SOD-1 conformers with conformer-specific monoclonal or polyclonal antibodies.

In another aspect, the present invention provides a method of determining whether an individual is predisposed to developing ALS, the method comprising: obtaining a cell free extract derived from cells or tissue taken from an individual; and identifying at least one SOD-1 conformer(s) in the cell free extract by one or more physical characteristics common to sporadic or familial ALS but distinctive from that of normal individuals. In some embodiments, such characteristics are selected from the group consisting of: immunological detection, electrophoretic mobility, and sedimentation rate. In other embodiments, the characteristics include differential reactivity to chemical reagents. In some embodiments, the individual has one or more ALS symptoms. In other embodiments, the individual has a family history of ALS. In still other embodiments, individual has a mutant SOD-1 protein. And in yet other embodiments, the immunological detection is determined using SOD-1 conformer-specific monoclonal or polyclonal antibodies.

In one exemplary embodiment of a diagnostic procedure in accordance with one embodiment of the invention, a patient presents to their physician, who after clinical examination concludes that ALS needs to be considered as a diagnosis. Currently this is a diagnosis of exclusion, meaning that after all other explanations for their symptoms are excluded, these patients are said to have ALS. But using the methods and materials provided by the present invention, ALS is no longer a diagnosis of exclusion; rather it can be addressed as soon as the clinical picture suggests the diagnosis. For example, under the situation presented a muscle biopsy would be performed on the patient and the tissue sample homogenized and subjected to our procedure of biotinylation and analysis by SDS PAGE and transfer to nitrocellulose for western blot analysis for the presence of the distinctive conformer of SOD-1 that is specific for familial and sporadic ALS. If the band is present, the patient can be said to have the disease. If absent, the disease is ruled out.

In still another aspect, the methods and materials provided by present invention provide a screen for small molecules that redirect SOD-1 biogenesis away from the pathway leading to the disease-associated conformer. An antibody has been raised that detects the putative earliest conform of the disease-associated conformer. By screening for small molecules that direct SOD-1 biogenesis away from this conformer (e.g., in a fluorescence capture plate assay give diminution of fluorescence when this conformer-specific antibody is biotinylated and its binding detected with neutravidin HRP), those having ordinary skill in the art can identify potential therapeutic agents.

These and other aspects and advantages will become apparent when the Description below is read in conjunction with the accompanying Drawings.

### 3 BRIEF DESCRIPTION OF THE DRAWINGS

FIG. 1 shows ALS disease-related SOD-1 conformers in cytosolic preparations of spinal cord from familial and sporadic ALS individuals. Cytosolic protein (10 μg) was electrophoresed in a 12% denatured cross-linked polyacrylamide gel (SDS) (Panel A) and a non-denatured cross-linked polyacrylamide gel (Panel B). Proteins in both gels were transferred to a membrane and immunoblotted using an rabbit antiserum raised against an SOD-1 peptide and that recognizes mouse and human wild-type SOD-1. Samples were from a transgenic mouse expressing human SOD-1 wild-type (Lane 1); sporadic ALS patients (Lanes 2, 4 and 5), familial ALS patient (A4V SOD-1 mutant) (Lane 3) familial ALS patient (G93C SOD-1 mutant) (Lanes 8 and 9); and a normal (control) individual (Lanes 6 and 7). The relative amount of the two conformers seen by this method in ALS patients is different from that of the control.

FIGS. 2A, 2B, 2C, and 2D show that cell-free translation products recapitulate the conformer heterogeneity observed in mutant vs. wild-type SOD-1 in familial and sporadic ALS vs. normal tissue. Each Figure shows cell-free translated SOD-1 mutant (G85R) and wild-type (WT) SOD-1 labeled with ³⁵S-cysteine were subjected to solution immunoprecipitation either under denaturing or non-denaturing conditions. Each figure depicts an autoradiograph of a 12% SDS-polyacrylamide gel. Lanes for total SOD-1 label in each assay (T); immunoprecipitated SOD-1 (P); and ¼ Of unbound SOD-1 (S) are identified. FIGS. 2A and 2B show immunoprecipitated material using the rabbit antiserum described in FIG. 1. FIG. 2A demonstrates that the antiserum preferentially recognizes mutant over wild-type SOD-1 under non-denaturing conditions while FIG. 2B demonstrates that the antiserum recognizes mutant and wild-type SOD-1 similarly under denaturing conditions. FIGS. 2C and 2D show immunoprecipitated material using a commercial murine monoclonal antibody to human SOD-1 (Sigma Chem. Co.). FIG. 2D demonstrates that the monoclonal antibody recognizes a similar fraction of wild-type and mutant SOD-1 under denaturing conditions while FIG. 2C demonstrates the antiserum recognizes a different fraction of wild-type and mutant SOD-1 under non-denaturing conditions. The overall results show that cell free synthesized wild-type and mutant SOD-1 form a variety of conformers which are differentially reactive with different anti-SOD-1 antibodies.

FIGS. 3A-3C illustrate the detection and identification of an ALS-specific form of SOD-1 in human spinal cord cytosol protein samples resulting from the experiments described in Sections 5.3.1 and 5.3.2 below. FIGS. 3A and 3B show the results of Western blot analysis of biotinylated (“+”) and non-biotinylated (“−”) proteins separated previously by SDS-PAGE. Multiple samples were used for each blot; and each blot had a common SALS-1 to which all blots were normalized. The 32 kD band indicates a protein signal specific for ALS (both SALS and FALS). The 20 kD band is labeled “hSOD-1”; the shift between the “+” and “−” blots is presumably due to the attachment of the biotin label. The two “Non-ALS” lanes are samples from a patient with Parkinson's Disease. FIG. 3C shows the relative intensities of biotinylated protein for normal vs. SALS vs. FALS (right-hand side) and ALS-afflicted vs. normal samples (left-hand side).

FIG. 4 is Western analysis of samples derived as described in Sections 5.3.1 and 5.3.2 below from protein samples taken from peripheral muscle and liver tissues. The spinal cord and liver samples were taken from a patient having SALS; the muscle sample was from a patient having FALS.

FIGS. 5A and 5B show an ALS-specific pattern of proteins is observed on a 2D gel after biotinylation as described in Sections 5.3.1 and 5.3.2 below. FIG. 5A shows the results from samples containing 150 μg of spinal cord cytosolic proteins before (upper panels) and after (lower panels) biotinylation were separated on 2D protein gels stained with SYPRO. The same protein molecular weight (MW) markers were used for all images. The amount of MW markers used for two upper images (indicated by yellow lines) was equal, and that used in two lower images (indicated by blue lines) was equal. FIG. 5B shows the quantification of the pattern of proteins. The total protein density of combined protein spots in the upper right panel normalized to the total density of protein MW markers was used for quantification. Five normal- and ten ALS-afflicted samples were included and the average value (±SEM) was also listed in the table (lower right). An asterisk (“*”) indicates that the difference between normal and ALS subjects was statistically significant, i.e., p=0.0005.

### 4 DESCRIPTION OF SOME EMBODIMENTS OF THE INVENTION

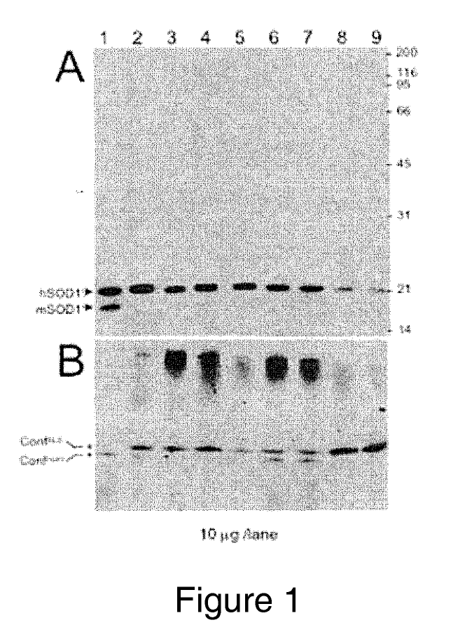

The present invention provides methods of diagnosing both familial and sporadic forms of ALS by identifying one or more disease-associated SOD-1 conformers or a disease-associated mixture of disease-associated SOD-1 conformers. In some cases, a disease-associated conformer may be unique to cytosol preparations of familial and/or sporadic ALS individuals. In other cases, two or more conformers present in ALS and normal cytosol may be present in ALS cytosol (sporadic and/or familial) in a ratio(s) that are unique when compared to the ratio(s) seen in cytosol of normal individuals. FIG. 1 shows an Example of the latter case.

4.1 ALS Diagnostic Assays

In one embodiment, the present invention provides a method of diagnosing ALS (sporadic or familial) that includes: obtaining a cell free extract derived from cells or tissue taken from an individual suspected of having sporadic or familial ALS; and identifying one or more SOD-1 conformer(s) in the cell free extract by one or more physical characteristics common to sporadic or familial ALS but distinctive from that of normal individuals. In some embodiments, such characteristics are selected from the group consisting of: immunological detection, electrophoretic mobility, and sedimentation rate. In other embodiments, the characteristics include differential reactivity to chemical reagents.

As used herein, an “individual suspected of having sporadic or familial ALS” is an individual with one or more ALS symptoms. Such an individual may also have a family history of ALS and may have a wild-type or a mutant SOD-1 protein sequence. Family history is preferably immediate family members including parents and siblings. Family history also may include grandparents.

As used herein, the phrase “cell-free extracts” refers to preparations from cells that comprise the intracellular contents of the cell. Such extracts are preferably substantially free of any intact or live cells. The intracellular contents may be nuclear, cytosolic or subfractions thereof. Cytosolic forms of cell-free extracts or further subfractions are preferred for use in the diagnostic methods of the invention. Cytosol can be prepared any technique known to those having ordinary skill in the art, including, but not limited to, homogenization and differential centrifugation both of which are well known in the art. For example, see Liu J et al., Neuron. 8;43(1):5-17 (2004). Additional methods of subcellular fractionation are well known and include precipitation, chromatography, and electrophoresis. Cell free extracts may be prepared from any human cells including blood cells (RBC or WBC), tissue samples (e.g., biopsy of muscle or skin), or cell containing body fluids, such as cerebrospinal fluid (CSF). Cells may be grown in tissue culture before testing. Samples may be processed from freshly isolated cells or from cells that have been previously stored under reduced temperature or frozen. Generally, cytosol is prepared by homogenization of cells followed by centrifugation at 100,000×g for one hour in order to remove intact cells, cell membranes and nuclei.

As used herein, “electrophoretic mobility” refers to the movement of a protein in an electrical field. The speed of travel is reflected in the position the protein has reached over time during application of the electrical field.

As used herein, “native gel electrophoresis” denotes to any form of gel electrophoresis that does not include denaturing agents. In general, the electrophoretic mobility of a particular protein in native gel electrophoresis reflects its mass, size, shape and charge. Denatured gel electrophoresis refers to electrophoresis conducted in the presence of one or more denaturing agents, such as sodium dodecyl sulfate, urea, and the like. A denaturing agent is one that modifies the three-dimensional structure of SOD-1 under the conditions used for electrophoresis. A variety of gels may be used in gel electrophoresis including cross-linked polyacrylamide, agarose, and the like.

As used herein, “immunological reactivity” refers to the ability of a protein to be detected by an antibody (or mixture of antibodies) under a particular set of conditions. Antibodies react with epitopes of proteins that may be linear or discontinuous. The ability of an antibody to detect or react with a protein is reflected in the affinity or avidity of binding.

As used herein, the term “antibody” includes immunoglobulins that are the product of B cells and variants thereof, as well as the T cell receptor (TcR) that is the product of T cells and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3 and IgG4 subclass.

A typical immunoglobulin structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The amino acids of an antibody may be natural or nonnatural.

Antibodies exist as full length intact antibodies or as a number of well-characterized fragments produced by digestion with various peptidases or chemicals. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the F(ab′)2 dimer into an Fab′ monomer. The Fab′ monomer is essentially a Fab fragment with part of the hinge region. (See FUNDAMENTAL IMMUNOLOGY, W. E. Paul, ed., Raven Press, N.Y. (1993), for a more detailed description of other antibody fragments.) While various antibody fragments are defined in terms of the digestion of an intact antibody, one of skill will appreciate that any of a variety of antibody fragments may be synthesized de novo either chemically or by utilizing recombinant DNA methodology. Thus, the term antibody, as used herein also includes antibody fragments either produced by the modification of whole antibodies or synthesized de novo or antibodies and fragments obtained by using recombinant DNA methodologies. Antibody fragments produced by recombinant techniques may include fragments known by proteolytic processing or may be unique fragments not available or previously known by proteolytic processing. Whole antibody and antibody fragments also may contain natural as well as unnatural amino acids. The term “antibody” also encompasses chimeric forms of antibody, CDR grafted antibody and other humanized forms of non-human antibodies.

Recombinant antibodies can include alterations in the amino acid sequence to provide for desired characteristics, for Example changes can be made in the variable region to provide improved antigen binding characteristics.

Immunological reactivity may involve antibodies that react with SOD-1 regardless of conformer type. Such antibodies may be reactive with a linear epitope that is characteristic in denatured SOD-1. Immunological reactivity also may be determined using conformer-specific monoclonal or polyclonal antibodies. SOD-1 conformer-specific monoclonal or polyclonal antibodies may be characterized by differential reactivity with SOD-1 prepared by in vitro synthesis of wild-type mRNA versus SOD-1 obtained by in vitro synthesis of mutant mRNA. The antibodies may react with a subset of in vitro synthesized wild-type SOD-1 and a different subset of in vitro synthesized mutant SOD-1; this may be observed simply as a difference in the proportion of input SOD-1 molecules bound by each antibody during immunoprecipitation. Differential reactivity may also arise when the antibody reacts with mutant in vitro synthesized SOD-1 but not wild-type in vitro synthesized SOD-1 (or vice versa).

SOD-1 conformer-specific monoclonal or polyclonal antibodies may be characterized by a substantial reduction or complete loss of the above described differential reactivity when immunological detection is evaluated using in vitro synthesized SOD-1 that has been denatured prior to antibody binding. This loss of differential reactivity may be manifest in an increase of reactivity with either wild-type or mutant in vitro synthesized SOD-1 or a decrease in reactivity. In either case, the result of the increase or decrease will be to equalize the proportion of SOD-1 detected for wild-type and mutant when denatured prior to evaluation.

Conformer specific monoclonal antibodies may be prepared by immunizing a mammal with recombinant human SOD-1. Specific SOD-1 conformers isolated by methods such as electrophoresis, density gradient ultracentrifugation, and the like, may be used for immunization. In particular, SOD-1 knockout mice provide a useful host for the preparation of monoclonal antibodies to SOD-1. SOD-1 knock-out mice may be prepared as described by Reaume et al., Nat Genet 13:43-47 (1996), or may be obtained commercially (e.g., from Cephalon, Inc. of Frazer, Pa.).

The route of administration can be intracutaneous subcutaneous, intramuscular, intraperitoneal or intravenous route and the method of administration is according to standard protocols known to those of skill in the art. Optionally, an adjuvant such as Freund's complete adjuvant, RIBI, alum or a recombinant cytokine such as interleukin-2 can be added or linked to the SOD-1 immunogen.

Monoclonal antibodies can be prepared in any number of ways known to those skilled in the art (see, for example, Kohler et al., Nature, 256: 495-497 (1975) and Eur. J. Immunol. 6:511-519 (1976); Milstein et al., Nature 266: 550-552 (1977), Koprowski et al., U.S. Pat. No. 4,172,124; Harlow, E. and D. Lane, 1988, ANTIBODIES: A LABORATORY MANUAL, Cold Spring Harbor Laboratory Press, Cold Spring Harbor, N.Y. (1989); CURRENT PROTOCOLS IN MOLECULAR BIOLOGY, Vol. 2 (Supplement 27, Summer '94), Ausubel, F. M. et al., Eds., (John Wiley & Sons: New York, N.Y.), Chapter 11, (1991)). Briefly, upon eliciting an immune response, the spleen is harvested, and splenocytes fused with myeloma cells by conventional methods. Resulting hybridomas can be screened by solution immunoprecipitation of SOD-1 cell-free translation products. Screening may also performed using solution immunoprecipitation of SOD-1 cell-free translation products enriched for individual conformers such as by native gel electrophoresis or immunoprecipitation using a conformer-specific polyclonal rabbit anti-SOD-1 peptide antisera (see Example 1 for details). Monoclonal antibodies differentially recognizing one SOD-1 conformer over another are identified from the generation of different patterns of conformer reactivity (different ratios of SOD-1 conformers) as seen in native vs denatured electrophoresis. Differences in the ratios of conformers immunoprecipitated or otherwise detected by different conformer-specific antibodies identified under native (non-denaturing) conditions generally disappear when binding is conducted under denaturing conditions.

As used herein, the term “immunoprecipitation” and the phrase “solution phase immunoprecipitation” refer to a process whereby a target soluble protein in a solution is removed from the solution following its binding by an antibody reactive with the protein. In general, the antibody is directly or indirectly bound to solid phase, which is contacted with the solution and later removed following binding of the target protein to the antibody. The target protein bound to the solid phase may be released and analyzed such as by gel electrophoresis. Depending on conditions, immunoprecipitation may remove some or all of the target protein in the solution.

Immunoprecipitation may be conducted using native or denatured forms of SOD-1 conformers. To immunoprecipitate denatured SOD-1, steps may be required to remove excess denaturing agent prior to antibody addition. For example, if SOD-1 conformers have been denatured with SDS (e.g., 100° C. for 2 min.), addition of a non-ionic detergent (e.g., triton X-100) can be used to enable subsequent immunoprecipitation analysis following antibody addition.

As used herein, “electrophoresis and detection with SOD-1 specific monoclonal or polyclonal antibodies” refers generally to any process that combines electrophoretic movement with immunological identity of the molecules subjected to the electrophoretic field. A preferred method is immunoblotting or “western blotting” where following gel electrophoresis, an electric field, applied at right angles to the first field, transfers the separated proteins to a membrane (e.g., nitrocellulose, PVP, etc.) which can be probed with an antibody for immunological reactivity. For example, see Liu J, et al., Neuron 8:43(1):5-17 (2004). Other approaches also may be used including simple incubation of the gel with the antibodies.

As used herein, the conformer physical characteristic of “sedimentation rate” refers to the rate at which a molecule sediments under zonal type density gradient ultracentrifugation. In zonal type density gradient ultracentrifugation, a macromolecular solution is carefully layered on top of a density gradient. Sucrose or glycerol is commonly used to form a density gradient for zonal ultracentrifugation. The sedimentation rate of a macromolecule is mainly a function of mass and shape.

During centrifugation, each species of macromolecule moves through the gradient at a rate largely determined by its sedimentation coefficient and therefore travels as a zone. After centrifugation, fractionation can be effected either by puncturing the bottom of the centrifuge tube or eluting from the top with a special pumping device.

Ratios of conformers may be used to diagnose ALS. SOD-1 conformer ratios may be determined by gel or blot scanning of individual conformers. Ratios from individuals suspected of having ALS can be compared to a standard curve of ratios from normal vs diseased (sporadic and familial) individuals.

In one embodiment of the diagnostic method, the cell free extract from an individual suspected of having ALS can be contacted with wild-type SOD-1 protein as it is synthesized by cell free translation. In this case, the cell-free translated SOD-1 conformers are evaluated for physical characteristics as described above. Although not wishing to be bound by any theory, increases in abnormal SOD-1 conformers or modified conformer ratios are believed to result from trans-acting factors present in the patient's cytosol.

As used herein, the phrase “contacted with . . . SOD-1 protein as it is synthesized by cell free translation” means that the agent is present during cell-free protein synthesis and has the potential to contact the nascent SOD-1 chain and to influence conformer formation.

In another aspect, the present invention provides a method of determining whether an individual is predisposed to ALS. The method can be applied to any individual. In particular, the method may be applied to individuals with a family history of ALS, to individuals that have only one or a few of the symptoms associated with ALS or have symptoms that are typical of ALS but are at an early stage such that ALS cannot be determined. The method also can be applied to individuals that have a mutant SOD-1 genetic sequence. In one approach, the method comprises identifying SOD-1 conformer(s) in the cell free extract by one or more physical characteristics common to sporadic or familial ALS but distinctive from that of normal individuals. In some embodiments, such characteristics are selected from the group consisting of: immunological detection, electrophoretic mobility, and sedimentation rate. In other embodiments, the characteristics include differential reactivity to chemical reagents. The same embodiments that have been described above for identification of physical characteristics of SOD-1 conformers are also applicable in this method of ALS predisposition determination.

As used herein, “symptoms associated with ALS” include at least one neurologically based symptom such as tripping and falling, loss of motor control in hands and arms, difficulty speaking, swallowing and/or breathing, persistent fatigue, and twitching and cramping.

4.2 Methods of Identifying SOD-1 Conformer Modulating Agents

The present invention also includes methods for identifying potential drug candidates that modulate SOD-1 conformer formation. Such assay can be utilized to identify small molecules, trans-acting factors signaling pathway inhibitors, and other agents that alter the distribution of SOD-1 conformers present in a cell. The method comprises contacting the agent with SOD-1 protein as it is synthesized by cell free translation and evaluating modulation of cell-free synthesized SOD-1 conformer formation by identifying one or more physical characteristics selected from the group consisting of: immunological detection, electrophoretic mobility, and sedimentation rate, which characterizes the different conformers. Thus, the various techniques of SOD-1 conformer detection described above in the diagnostic and predisposition assays are also applicable to the agent screening assay.

The nucleotide sequence of human SOD-1 cDNA is available from GenBank under accession no. NM_{—}000454 (Swiss Prot accession no. P00441) (SEQ ID NO:2). The SOD-1 gene contains five exons and encodes a 153-amino acid polypeptide encoded by nucleotides at positions 149-613 of the mRNA. SOD-1 cDNA can be obtained by RT-PCR amplification from genomic DNA using suitable enzymes and primers.

SOD-1 encoding nucleic acid may be cloned into an appropriate transcription vector for preparing SOD-1 mRNA. The cDNA is inserted into the vector under control of a prokaryotic promoter for a DNA dependent bacteriophage RNA polymerase such as SP6, T3 and T7. Cell-free transcription reactions can be conducted under standard protocols, generally containing vector, Tris buffer MgCl2, Spermidine, rNTPs, RNA polymerase and Rnase inhibitor.

Cell-free translation from purified SOD-1 mRNA may be performed using wheat germ extract (WGE) or rabbit reticulocyte lysate (RRL) as is well known in the art. WGL and RRL materials are commercially available (e.g., Ambion) or may be prepared as described herein. Transcription-linked-translation approaches that can utilize unpurified mRNA from a transcription reaction can also be used. The “linked” system is a two-step reaction, based on transcription with a bacteriophage polymerase followed by translation in the RRL or WGE. Because the transcription and translation reactions are separate, each can be optimized to ensure that both are functioning at their full potential.

As described above, candidate agents are added to the cell-free translation system so that they are present and can interact with conformer formation by nascent SOD-1. Once translation is completed, individual SOD-1 conformers are detected by immunological detection, electrophoretic mobility, and sedimentation rate using the same approaches as described for the diagnostic assays. The types of conformers visualized following cell-free synthesis of wild-type or mutant forms of SOD-1 in the absence of the agent are compared with the types of conformers seen in the presence of the agent. An agent that changes the amount of a particular SOD-1 conformer or the relative ratios of SOD-1 conformers modulates SOD-1 conformer formation.

Candidate SOD-1 conformer modulating agents are generally small molecule organic compounds of 5,000 daltons or less such as drugs, proteins, peptides, peptidomimetics, glycoproteins, proteoglycans, lipids glycolipids, phospholipids, lipopolysaccharide, nucleic acids, proteoglycans, carbohydrates, and the like.

The following examples serve to illustrate the present invention. These examples are in no way intended to limit the scope of the invention.

### 5 EXAMPLES

The following Examples are provided to illustrate certain aspects of the present invention and to aid those of skill in the art in the art in practicing the invention. These Examples are in no way to be considered to limit the scope of the invention in any manner.

### 5.1 Example 1

### Diagnostic Assay for Sporadic and Familial ALS

Cytosol was obtained from human spinal cord essentially as described by Liu (2004).

SOD-1 in the cytosol was screened by native crosslinked polyacrylamide gel and denatured crosslinked polyacrylamide gel (SDS) electrophoresis and immunoblotted using a rabbit antiserum prepared against SOD-1 peptide and which reacts with both mouse and human wild-type SOD-1 under denaturing conditions but only human SOD-1 under native conditions. The rabbit was immunized with the peptide: NH₂-CYDDLGKGGNEESTK-COOH (SEQ ID NO:1) conjugated to keyhole limpet hemocyanin (KLH) as previously described by Pardo et al., Proc Natl Acad Sci USA. 14;92(4):954-8 (1995).

As shown in FIG. 1, the normal cell extract contained a pair of SOD-1 conformers observed by immunoblotting of the native gel (panel B). Immunoblotting was performed as described previously (see, e.g., Liu J, et al., Neuron 8;43(1):5-17 (2004)). However, in sporadic and familial ALS cytosols, the antibody detected mainly a single conformer in the native gel, apparently identical in both ALS samples and identical to one of the SOD-1 conformers observed in the normal cytosol. Thus, the results show that spinal cord cytosol from sporadic and familial forms of ALS contain a similar ratio of SOD-1 conformers that is distinct from the ratio of conformers present in cytosol of normal spinal cord.

The SOD-1 conformer characteristic of sedimentation rate was determined by glycerol zonal density gradient ultracentrifugation. For example, solutions of 50 mM Hepes pH 7.6 50 mM potassium acetate 1 mM DTT, 1 mM PMSF, 0.2% Triton and either 0% or 30% glycerol were used to form a linear gradient from 10%-30% glycerol. The gradient was chilled to 4° C. and over-layered with 50 μl of sample and subjected to ultracentrifugation in a Beckman TL-100 table top ultracentrifuge in the TLS-55 Rotor at 55,000 rpm for 16 hrs. Gradients were fractionated in 100 μl aliquots from the top and 15 μL analysed by SDS-PAGE. The effect of total cytosol or specific proteins or intrinsic size/shape/coassociation differences between mutants of SOD-1 are assessed by relative migration in the gradient.

### 5.2 Example 2

### Method for Identifying ALS Therapeutic Agents

5.2.1 Cell-Free Translation of SOD-1

To prepare SOD-1 mRNA, human SOD-1 cDNA, obtained from D. Borcheldt (see Ratovitski et al., Hum Mol. Genet. 8: 1451-1460 ((1999)), was subcloned in genetic linkage with the SP6 promoter in Bluescript vector (Stratagene). The expression plasmid was linearized downstream of the termination codon by digesting with BamH1. Cell-free transcription reactions were prepared containing 0.2 mg/ml plasmid DNA, 40 mM Tris pH 7.9, 6 mM MgCl₂, 2 mM Spermidine, 0.5 mM of each NTP, and 1 unit each of SP6 polymerase and Rnase inhibitor per 2.5 μL of transcription reaction. Transcription was performed at 40° C. for 1 hr, and transferred to ice upon completion.

Transcription-linked translation reactions were prepared by adding SOD-1 transcription reaction product at 20% of the final translation volume. Also added was ATP and GTP at 1 mM each, creatine phosphate at 10 mM and amino acids at 40 μM. The total salt concentration in the translation reaction taking into account the salt from the transcription reaction and salt from wheat germ extract was 4 mM Mg and 100 mM potassium acetate. Twenty percent (20%) of the translation volume consisted of wheat germ extract. Translation was carried out at 26° C. for 60 minutes and terminated by placement on ice.

Wheat germ extract was prepared by grinding 11 grams fresh wheat germ in 14 ml buffer containing 40 mM Hepes pH 7.8, 100 mM potassium acetate, 1 mM Magnesium acetate, 2 mM calcium chloride, 4 mM dithiothreitol, centrifugation at 23,000×g/15 minutes with the resulting extract exchanged into 25 mM Hepes pH 7.8, 100 mM potassium acetate, 5 mM magnesium acetate, 4 mM dithiothreitol by centrifuge desalting on G-25 fine Sephadex spin columns. The excluded volume from the spin columns, roughly equivalent to the applied volume, was centrifuged at 23,000×g/15 minutes and the supernatant carefully collected, mixed, aliquoted and frozen at −80° C. ³⁵S-cysteine (Amersham; 1,000 Ci/mmole 10 μCi/μL) was added to 1 μL per 20 μl translation reaction. The other 19 amino acids were at 40 μM final concentration.

Translation in the rabbit reticulocyte (RRL) cell-free system was performed as above except that RRL constituted 35% of the translation reaction volume as compared with 20% for the wheat germ cell-free system. RRL was prepared as described previously by Shields and Blobel, J. Biol. Chem. 252: 1592-1596 (1979).

5.2.2 Conformer Detection in Cell-Free Translated SOD-1

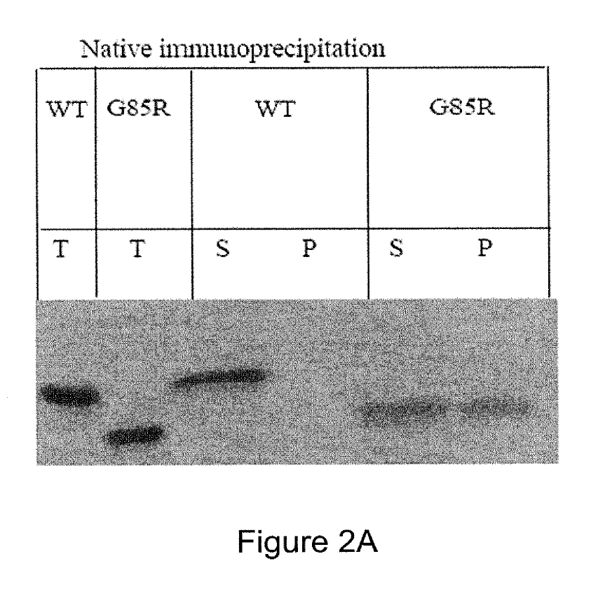

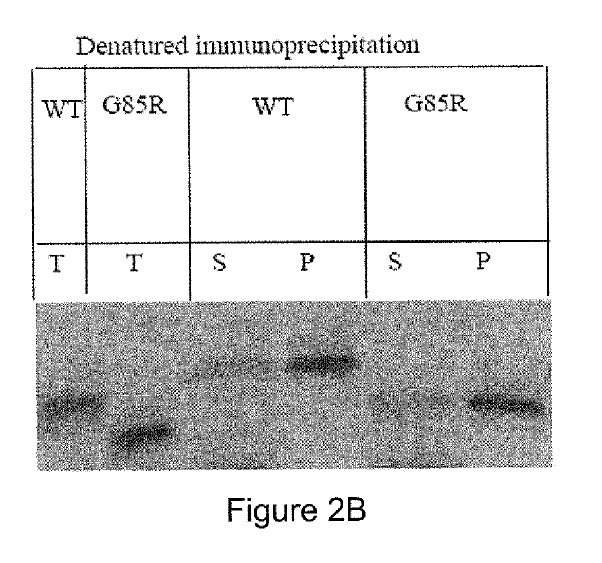

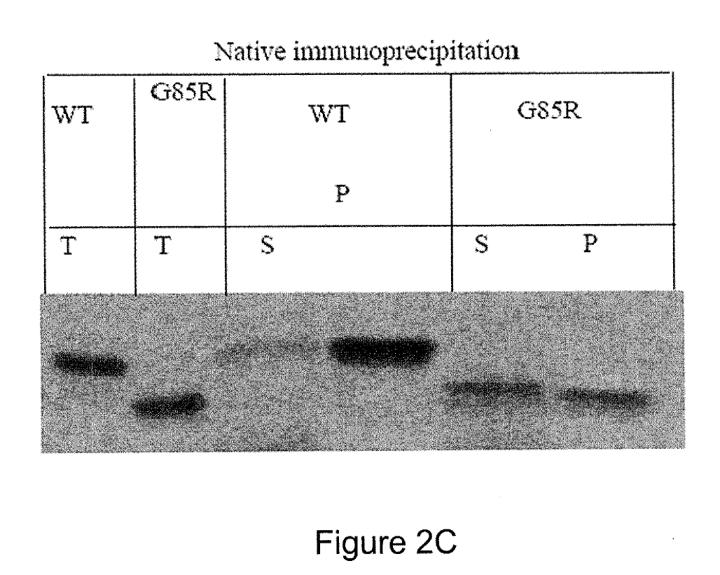

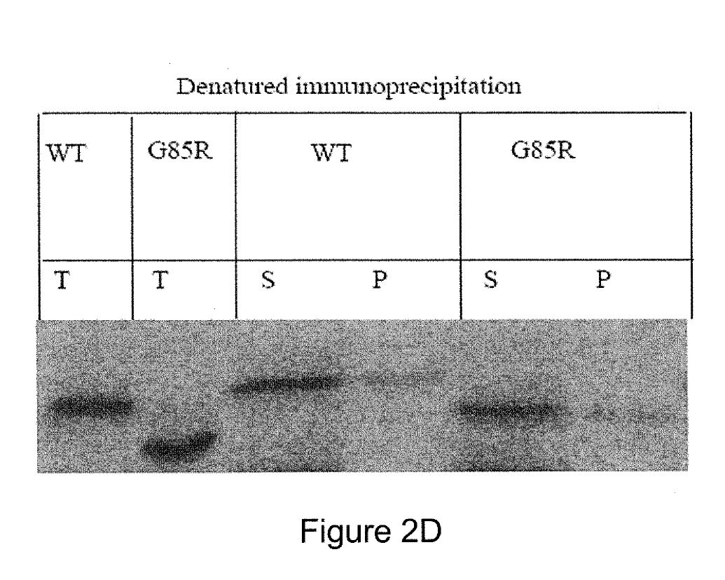

FIGS. 2A-2D depict autoradiographs of ³⁵S-cysteine labeled SOD-1 conformers translated as described above. Conformer heterogeneity was observed in both native and denatured solution phase immunoprecipitation with SOD-1 antibodies. Details for solution phase immunoprecipitation are described in Chuck S L, et al., Cell 68:9-21 (1992). In brief, samples were diluted 20-fold with 1% Triton, 100 mM Tris pH 8.0, 100 mM sodium chloride, 10 mM EDTA, 1 mM PMSF and 1 μl serum or up to 1 μg of monoclonal antibody and Protein A-Sepharose beads were added (7.5 μL-packed beads per microliter of serum). The mixture was incubated 12 hrs at 4° C. Beads were washed three times and antibody-bound proteins eluted by boiling in SDS-PAGE sample buffer.

FIGS. 2A and 2B shows material immunoprecipitated using the anti-SOD-1 peptide rabbit antiserum as described above for FIG. 1. FIGS. 2C and 2D shows immunoprecipitation using a commercial murine monoclonal antibody to human SOD-1 raised against recombinant SOD-1 (Sigma Chem. Co., product # S2147). The results from both panels show that multiple SOD-1 conformers can be identified in both wild-type and mutant SOD-1 cell free translation products. The proportion of conformers that react with each antibody under denatured or non-denatured immunoprecipitation varied for wild-type and mutant SOD-1 translation products.

5.2.3 Identifying Agents that Modulate SOD-1 Conformer Formation

Agents to be tested as modulators of SOD-1 conformer formation are identified using cell-free translated SOD-1 as described directly above. Various concentrations of the agent are added to the translation mixture so that the agent is present during cell-free synthesis. The types of SOD-1 conformers are identified using solution phase immunoprecipitation with antiserum or monoclonal antibody under denatured or native conditions for a control (no agent added) and for each candidate modulating agent.

The types of conformers visualized following cell-free synthesis of wild-type or mutant forms of SOD-1 in the absence of the agent are compared with the types of conformers seen in the presence of the agent. An agent that changes the amount of a particular SOD-1 conformer or the relative ratios of SOD-1 conformers modulates SOD-1 conformer formation.

### 5.3 Example 3

### Different SOD-1 Conformers Correspond to Sporadic and Familial ALS

5.3.1 Conformer-Specific Modification of Proteins

We decided to detect protein structural differences that result in differences in modifications by chemical reagents (Soares and Giglio, 2003; Goldberg et al., 2003). Specifically, chose to utilize a cross-linking reagent that conjugates biotin to many proteins. The reagent used for the proposed study is sulfo-N-hydroxysuccinimide-Long Chain-biotin (sulfo-NHS-LCbiotin, below).

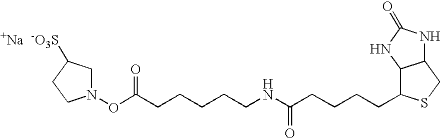

This compound reacts with primary amino groups (—NH₂) in pH7-9 buffers to form amide-bound detectably labeled proteins:

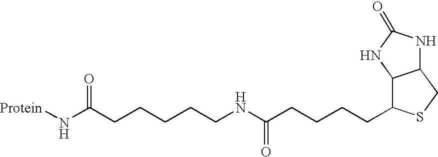

Without being bound to any specific theory of action, the extent to which a protein can be modified using this method depend upon the available primary amine moieties in the protein conformation. Thus, differences in the protein's three-dimensional structure (e.g., the protein's fold) may alter the availability of available primary amino groups to the sulfo-NHS-LC-biotin reagent; thereby resulting in different types or degrees of modified proteins (or both). The modified proteins can be detected via Western analysis and/or 2D protein electrophoresis.

5.3.2 An SOD-1 Conformer is a Biomarker for FALS

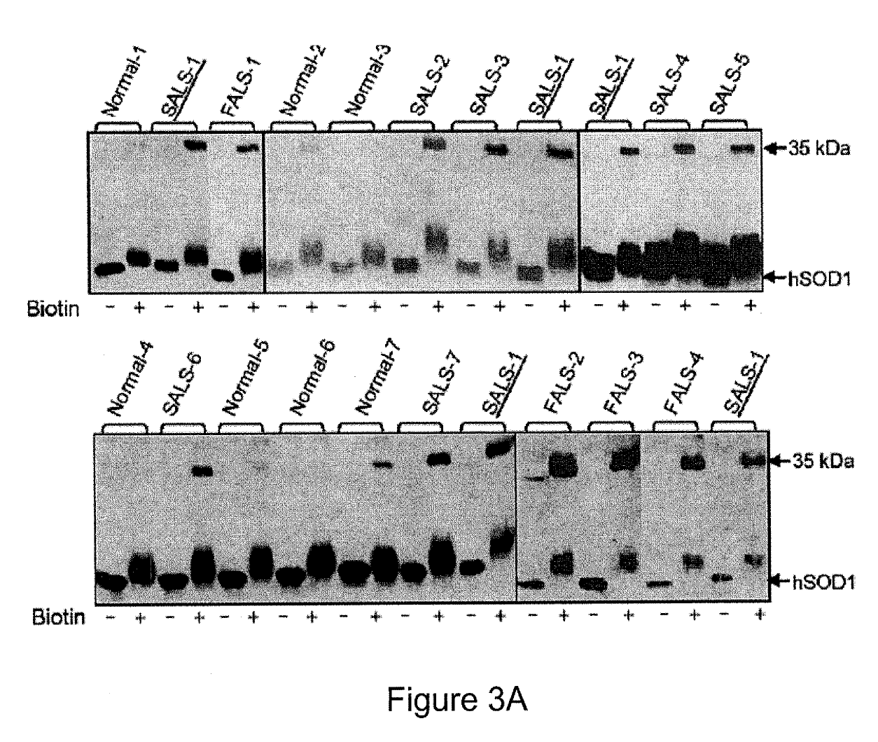

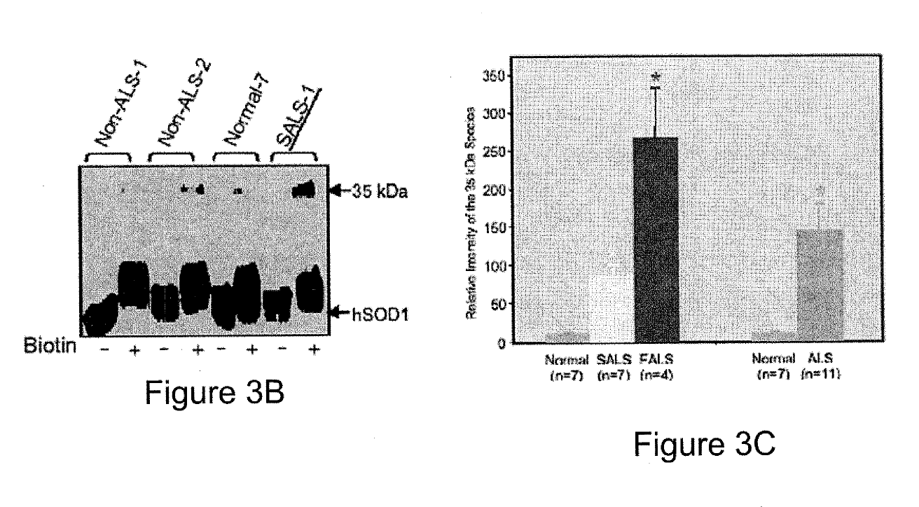

Cytosolic proteins taken from the spinal cord material of normal subjects, ALS-afflicted subjects, and subjects having known neurological diseases other than ALS were prepared as described (Liu et al., 2004). A total of 5- to 10 μg of cytosolic proteins were incubated with 10 mM sulfo-NHS-LC-biotin (Pierce) in PBS buffer at 28° C. for 23 min. The reaction was stopped by adding 20 mM lysine and incubated for another 20 min. at the same temperature. Biotin-modified proteins were separated on SDS-PAGE, transferred to the nitrocellulose membrane, and probed with an antibody against SOD-1 (Pardo et al., 1995) for Western analysis. The results are presented in FIG. 3. As expected, the human SOD-1 protein signal in absence of biotin (FIG. 3A, indicated by an arrow labeled as “hSOD-1”, in “−” lanes) was shifted to a slightly higher molecular weight band (FIG. 3A, in “+” lanes), presumably due to addition of the biotin moieties. Furthermore, a 32 kDa protein band that is immunoreactive with the SOD-1 antibody was present specifically in the spinal cords of ALS, but not normal or non-ALS subjects (FIGS. 3A, and 3B, in “+” lanes and indicated by an arrow labeled as “35 kDa”). Pre-incubation of the primary antibody solution with the peptide to which the antibody was made against abolished the 32 kDa signal (data not shown) indicating that this protein species is a SOD-1-related molecule. Quantitatively, the amount of 32 kDa signal is comparable between FALS caused by mutations in the SOD-1 gene and SALS (FIG. 3C). FALS or SALS samples differed significantly from the normal samples with regard to the intensity of the 32 kDa band (p=0.0004, indicated by the yellow *, and p=0.0015, indicated by the red *, respectively). Together, there is about 14-fold increase of this modified SOD-1-immunoreactive protein species in ALS samples compared to the normal samples (FIG. 3C, p=0.012, indicated by the orange *).

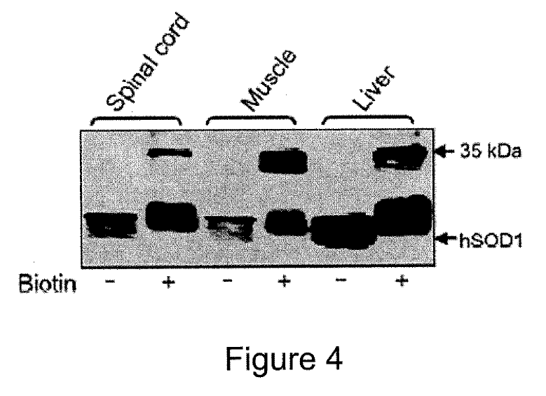

ALS-specific 32 kDa protein species was also observed in peripheral tissues, both muscle and liver, the two peripheral tissues currently available. The same protein-species was highly abundant in both muscle and liver tissues from ALS patients. See FIG. 4. Nanoelectrospray tandem mass spectrometry was performed on the biotinylated ALS-specific band and its identity as SOD-1 was confirmed.

5.3.3 A Unique Pattern of Biotinylated Proteins is Observed in ALS Spinal Cord Via 2D Protein Electrophoresis

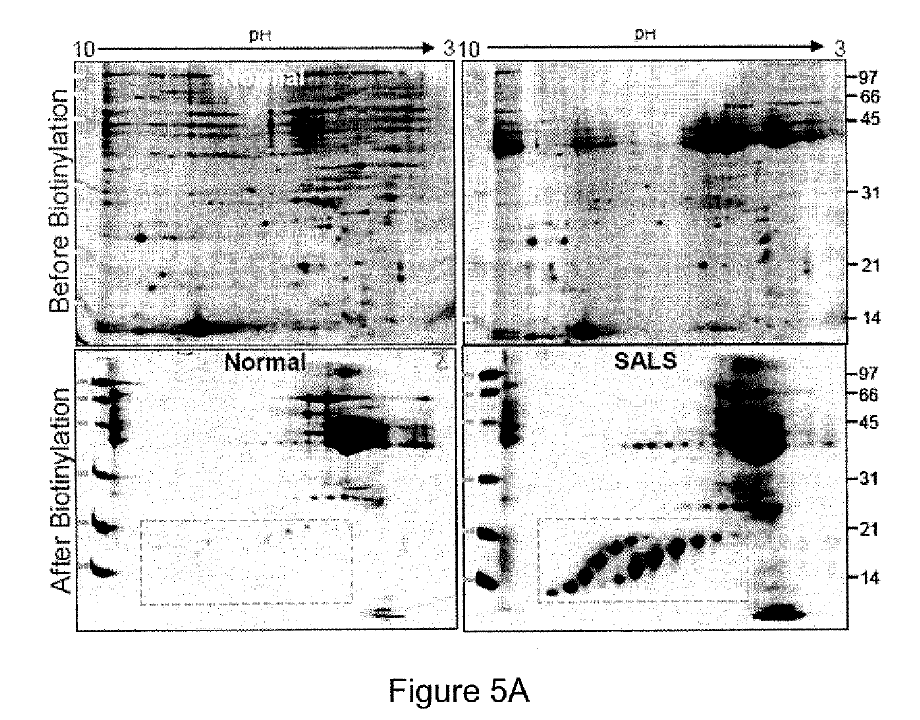

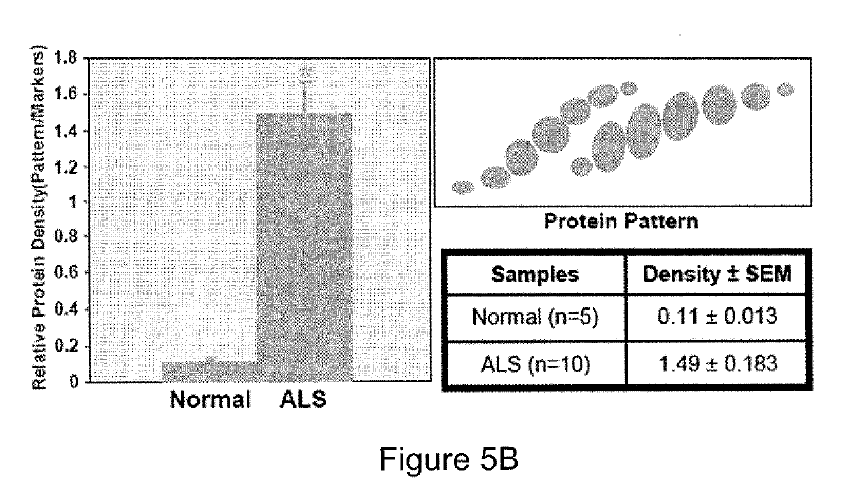

In order to elucidate possible differences in the conformation of other proteins in ALS spinal cord, potential ALS-specific protein patterns were revealed by biotinylation using 2D protein electrophoresis. Protein samples were similarly prepared as described above (Sections 5.3.1) with a few modifications. A total of 150 μg of proteins were used for biotinylation. Biotinylated protein samples were de-lipidated using ReadyPrep 2-D Cleaning Kit (BioRad). Precipitated proteins were resuspended in ReadyPrep Rehydration buffer (BioRad). Proteins were loaded on the 7 cm IPG strips (BioRad, pH 3-10) and separated via Isoelectric Focusing (IEF) electrophoresis system, followed by the 2nd dimensional separation on 12% SDS-PAGE gels. Gels were fixed in 10% methanol/7% acidic acid and stained with SYPRO-Ruby (Molecular Probes). Stained gels were imaged on Fluorchem 8900 imaging System (Alpha Innotech). A unique pattern of biotinylated protein spots was observed with all ALS spinal cord samples tested so far, but largely absent in normal samples. The data obtained is shown in FIG. 5A (compare the area outlined by the perforated yellow box, in Normal and SALS blots, respectively). FIG. 5B shows the quantification of the pattern of proteins.

### 6 EMBODIMENTS

From the foregoing, it will be appreciated that the present invention can be expressed in a variety of embodiments. Some exemplary embodiments include:
1. A method of diagnosing ALS, comprising detecting an SOD-1 biomarker correlated with the presence of ALS in a patient.
2. The method of embodiment 1, further comprising isolating a sample from the peripheral tissue of the patient.
3. The method of embodiment 2, wherein the isolating includes collecting a sample from the peripheral muscle, liver, or spinal fluid of the patient.
4. The method of embodiment 3 or embodiment 2, further comprising biotinylating the SOD-1 biomarker.
5. A polypeptide having the sequence: CYDDLGKGGNEESTK (SEQ ID NO:1).
6. An antibody having substantially specific affinity to an SOD-1 conformer that is associated with the presence of ALS in a patient.
7. The antibody of embodiment 6, wherein the conformer is associated with the presence of SALS.
8. The antibody of embodiment 6, wherein the conformer is associated with the presence of FALS.
9. A method of diagnosing whether an individual has sporadic or familial ALS, the method comprising:
   obtaining a cell free extract derived from cells or tissue taken from an individual suspected of having sporadic or familial ALS; and
   identifying SOD-1 conformer(s) in the cell free extract by one or more physical characteristics selected from the group consisting of: immunological detection, electrophoretic mobility, and sedimentation rate, said one more physical characteristics being common to sporadic or familial ALS but distinctive from that of normal individuals.
10. The method of embodiment 9 wherein immunological detection is determined using SOD-1 conformer-specific monoclonal or polyclonal antibodies.
11. The method of embodiment 10 wherein conformer-specific monoclonal or polyclonal antibodies are characterized by differential reactivity with SOD-1 prepared by in vitro synthesis of wild-type mRNA versus SOD-1 obtained by in vitro synthesis of mutant mRNA.
12. The method of embodiment 11 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
13. The method of embodiment 10 wherein conformer-specific monoclonal or polyclonal antibodies are characterized by differential reactivity shown by binding with mutant in vitro synthesized SOD-1 but not wild-type in vitro synthesized SOD-1, or vice versa.
14. The method of embodiment 13 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
15. The method of embodiment 9 wherein immunological detection comprises immunoprecipitation of SOD-1 conformers with conformer-specific monoclonal or polyclonal antibodies.
16. The method of embodiment 9 wherein electrophoretic mobility is determined using native gel electrophoresis.
17. The method of embodiment 9 wherein step b) is achieved by a combination of electrophoretic mobility and immunological detection.
18. The method of embodiment 9 wherein the characteristic of sedimentation rate is determined using density gradient ultracentrifugation.
19. The method of embodiment 9 wherein the cell free extract is contacted with wild-type SOD-1 protein as it is synthesized by cell free translation, and wherein the evaluated SOD-1 conformers are the cell-free translated SOD-1 conformer(s).
20. The method of embodiment 9 wherein the cell free extract is a cytosolic preparation.
21. The method of embodiment 9 wherein the cells are from cerebrospinal fluid.
22. The method of embodiment 9 wherein the cells are blood cells.
23. The method of embodiment 9 wherein the tissue is spinal cord.
24. The method of embodiment 9 wherein two or more SOD-1 conformers are identified.
25. The method of embodiment 24 wherein a ratio of the two or more SOD-1 conformers in the cell extract is obtained and wherein said ratio is common to sporadic and familial ALS but distinctive from that of normal individuals.
26. The method of embodiment 9 wherein said individual has one or more symptoms associated with ALS.
27. A method of determining whether an individual is predisposed to developing ALS, the method comprising:
   obtaining a cell free extract derived from cells or tissue taken from an individual, and
   identifying SOD-1 conformer(s) in the cell free extract by one or more physical characteristics selected from the group consisting of: immunological detection, electrophoretic mobility, and sedimentation rate, said one more physical characteristics being common to sporadic or familial ALS but distinctive from that of normal individuals.
28. The method of embodiment 27 wherein said individual has one or more ALS symptoms.
29. The method of embodiment 27 wherein said individual has a family history of ALS.
30. The method of embodiment 27 wherein said individual has a mutant SOD-1 protein.
31. The method of embodiment 27 wherein immunological detection is determined using SOD-1 conformer-specific monoclonal or polyclonal antibodies.
32. The method of embodiment 31 wherein conformer-specific monoclonal or polyclonal antibodies are characterized by differential reactivity with SOD-1 prepared by in vitro synthesis of wild-type mRNA versus SOD-1 obtained by in vitro synthesis of mutant mRNA.
33. The method of embodiment 32 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
34. The method of embodiment 31 wherein conformer-specific monoclonal or polyclonal antibodies are characterized by differential reactivity shown by binding with mutant in vitro synthesized SOD-1 but not wild-type in vitro synthesized SOD-1, or vice versa.
35. The method of embodiment 34 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
36. The method of embodiment 27 wherein electrophoretic mobility is determined using native gel electrophoresis.
37. The method of embodiment 27 wherein step b) is achieved by a combination of electrophoretic mobility and immunological detection.
38. The method of embodiment 27 wherein the characteristic of sedimentation rate is determined using density gradient ultracentrifugation.
39. The method of embodiment 27 wherein the cell free extract is contacted with wild-type SOD-1 protein as it is synthesized by cell free translation, and wherein the evaluated SOD-1 conformers are the cell-free translated SOD-1 conformer(s).
40. The method of embodiment 27 wherein the cell free extract is a cytosolic preparation.
41. The method of embodiment 27 wherein the cells are from cerebrospinal fluid.
42. The method of embodiment 27 wherein the cells are blood cells.
43. The method of embodiment 27 wherein the tissue is spinal cord.
44. The method of embodiment 27 wherein two or more SOD-1 conformers are identified.
45. The method of embodiment 36 wherein a ratio of the two or more SOD-1 conformers in the cell extract is obtained and wherein said ratio is common to sporadic and familial ALS but distinctive from that of normal individuals.
46. A method of identifying an agent that modulates SOD-1 conformer formation, the method comprising contacting the agent with SOD-1 protein as it is synthesized by cell free translation and evaluating modulation of cell-free synthesized SOD-1 conformer formation by identifying one or more physical characteristics selected from the group consisting of: immunological detection, electrophoretic mobility, and sedimentation rate.
47. The method of embodiment 46 wherein immunological detection is determined using SOD-1 conformer-specific monoclonal or polyclonal antibodies.
48. The method of embodiment 47 wherein conformer-specific monoclonal or polyclonal antibodies are characterized by differential reactivity with SOD-1 prepared by in vitro synthesis of wild-type mRNA versus SOD-1 obtained by in vitro synthesis of mutant mRNA.
49. The method of embodiment 48 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
50. The method of embodiment 49 wherein conformer-specific monoclonal or polyclonal antibodies are characterized by differential reactivity shown by binding with mutant in vitro synthesized SOD-1 but not wild-type in vitro synthesized SOD-1, or vice versa.
51. The method of embodiment 50 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.
52. The method of embodiment 46 wherein electrophoretic mobility is determined using native gel electrophoresis.
53. 45. The method of embodiment 46 wherein step b) is achieved by a combination of electrophoretic mobility and immunological detection.
54. The method of embodiment 46 wherein the characteristic of sedimentation rate is determined using density gradient ultracentrifugation.
55. The method of embodiment 46 wherein contacting between the agent and SOD-1 protein as it is synthesized by cell free translation further includes contacting with a cell free extract from cells or tissue of an individual with familial or sporadic ALS.
56. The method of embodiment 47 wherein the cell free extract is a cytosolic preparation.
57. The method of embodiment 47 wherein the cells are from cerebrospinal fluid.
58. The method of embodiment 47 wherein the cells are blood cells.
59. The method of embodiment 47 wherein the tissue is spinal cord.
60. The method of embodiment 46 wherein the SOD-1 protein synthesized by cell free translation is a wild-type SOD-1 protein.
61. The method of embodiment 46 wherein the SOD-1 protein synthesized by cell free translation is a mutant SOD-1 protein.
62. The method of embodiment 46 wherein two or more SOD-1 conformers are synthesized by cell free translation.
63. The method of embodiment 53 wherein the relative amounts of the two or more SOD-1 conformers is determined and a ratio obtained, and wherein modulation of SOD-1 conformer formation is identified by a change in the ratio of conformers.
64. The method of embodiment 46 wherein the agent is an organic molecule of less than 5,000 daltons.

### 7 CONCLUSION

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### 8 BIBLIOGRAPHY

The following references are incorporated herein by reference in their entirety and for all purposes.
Chen, Y. Z., Bennett, C. L., Huynh, H. M., Blair, I. P., Puls, I., Irobi, J., Dierick, I., Abel, A., Kennerson, M. L., Rabin, B. A., et al. (2004). DNA/RNA helicase gene mutations in a form of juvenile amyotrophic lateral sclerosis (ALS4). Am J Hum Genet 74, 1128-1135.
Clement, A. M., Nguyen, M. D., Roberts, E. A., Garcia, M. L., Boillee, S., Rule, M., McMahon, A. P., Doucette, W., Siwek, D., Ferrante, R. J., et al. (2003). Wild-type nonneuronal cells extend survival of SOD-1 mutant motor neurons in ALS mice. Science 302, 113-117.
Goldberg, N. R., Beuming, T., Soyer, O. S., Goldstein, R. A., Weinstein, H., and Javitch, J. A. (2003). Probing conformational changes in neurotransmitter transporters: a structural context. Eur J Pharmacol 479, 3-12.
Gooch, C. L., and Shefner, J. M. (2004). ALS surrogate markers. MUNE. Amyotroph Lateral Scler Other Motor Neuron Disord 5 Suppl 1, 104-107.
Hadano, S., Hand, C. K., Osuga, H., Yanagisawa, Y., Otomo, A., Devon, R. S., Miyamoto, N., Showguchi-Miyata, J., Okada, Y., Singaraja, R., et al. (2001). A gene encoding a putative GTPase regulator is mutated in familial amyotrophic lateral sclerosis 2. Nat Genet 29, 166-173.
Kalra, S., and Arnold, D. L. (2004). ALS surrogate markers. MRS. Amyotroph Lateral Scler Other Motor Neuron Disord 5 Suppl 1, 111-114.
Kaufmann, P., and Mitsumoto, H. (2002). Amyotrophic lateral sclerosis: objective upper motor neuron markers. Curr Neurol Neurosci Rep 2, 55-60.
Liu, J., Lillo, C., Jonsson, P. A., Vande Velde, C., Ward, C. M., Miller, T. M., Subramaniam, J. R., Rothstein, J. D., Marklund, S., Andersen, P. M., et al. (2004). Toxicity of familial ALS-linked SOD-1 mutants from selective ecruitment to spinal mitochondria. Neuron 43, 5-17.
Majoor-Krakauer, D., Willems, P. J., and Hofman, A. (2003). Genetic epidemiology of amyotrophic lateral sclerosis. Clin Genet 63, 83-101.
Nishimura, A. L., Mitne-Neto, M., Silva, H. C., Richieri-Costa, A., Middleton, S., Cascio, D., Kok, F., Oliveira, J. R., Gillingwater, T., Webb, J., et al. (2004). A mutation in the vesicle-trafficking protein VAPB causes late-onset spinal muscular atrophy and amyotrophic lateral sclerosis. Am J Hum Genet 75, 822-831.
Pardo, C. A., Xu, Z., Borchelt, D. R., Price, D. L., Sisodia, S. S., and Cleveland, D. W. (1995). Superoxide dismutase is an abundant component in cell bodies, dendrites, and axons of motor neurons and in a subset of other neurons. Proc Natl Acad Sci USA 92, 954-958.
Rachakonda, V., Pan, T. H., and Le, W. D. (2004). Biomarkers of neurodegenerative disorders: how good are they? Cell Res 14, 347-358.
Rosen, D. R., Siddique, T., Patterson, D., Figlewicz, D. A., Sapp, P., Hentati, A., Donaldson, D., Goto, J., O'Regan, J. P., Deng, H. X., and et al. (1993). Mutations in Cu/Zn superoxide dismutase gene are associated with familial amyotrophic lateral sclerosis. Nature 362, 59-62.
Ross, C. A., and Poirier, M. A. (2004). Protein aggregation and neurodegenerative disease. Nat Med 10 Suppl, S10-17.
Simpson, E. P., Henry, Y. K., Henkel, J. S., Smith, R. G., and Appel, S. H. (2004). Increased lipid peroxidation in sera of ALS patients: a potential biomarker of disease burden. Neurology 62, 1758-1765.
Soares, A. M., and Giglio, J. R. (2003). Chemical modifications of phospholipases A2 from snake venoms:effects on catalytic and pharmacological properties. Toxicon 42, 855-868.
Strong, M., and Rosenfeld, J. (2003). Amyotrophic lateral sclerosis: a review of current concepts. Amyotroph Lateral Scler Other Motor Neuron Disord 4, 136-143.
Williams, D. B., and Windebank, A. J. (1991). Motor neuron disease (amyotrophic lateral sclerosis). Mayo Clin Proc 66, 54-82.
Winhammar, J. M., Rowe, D. B., Henderson, R. D., and Kiernan, M. C. (2005). Assessment of disease progression in motor neuron disease. Lancet Neurol 4, 229-238.
Yang, Y., Hentati, A., Deng, H. X., Dabbagh, O., Sasaki, T., Hirano, M., Hung, W. Y., Ouahchi, K., Yan, J., Azim, A. C., et al. (2001). The gene encoding alsin, a protein with three guanine-nucleotide exchange factor domains, is mutated in a form of recessive amyotrophic lateral sclerosis. Nat Genet 29, 160-165.

What is claimed:

1. A method of diagnosing amyotrophic lateral sclerosis (ALS), comprising detecting a copper-zinc superoxide dismutase-1 (SOD-1) biomarker correlated with the presence of ALS in a patient.

2. The method of claim 1, further comprising isolating a sample from the peripheral tissue of the patient.

3. The method of claim 2, wherein the isolating includes collecting a sample from the peripheral muscle, liver, or spinal fluid of the patient.

4. The method of claim 3 or claim 2, further comprising biotinylating the SOD-1 biomarker.

5. A polypeptide having the sequence: CYDDLGKGGNEESTK (SEQ ID NO:1).

6. An antibody having substantially specific affinity to an SOD-1 conformer that is associated with the presence of ALS in a patient.

7. The antibody of claim 6, wherein the conformer is associated with the presence of sporadic ALS.

8. The antibody of claim 6, wherein the conformer is associated with the presence of familial ALS.

9. A method of diagnosing whether an individual has sporadic or familial ALS, the method comprising:
obtaining a cell free extract derived from cells or tissue taken from an individual suspected of having sporadic or familial ALS; and
identifying SOD-1 conformer(s) in the cell free extract by one or more physical characteristics common to sporadic or familial ALS but distinctive from that of normal individuals.

10. The method of claim 9 wherein immunological detection is determined using SOD-1 conformer-specific monoclonal or polyclonal antibodies.

11. The method of claim 10 wherein conformer-specific monoclonal or polyclonal antibodies are characterized by differential reactivity with SOD-1 prepared by in vitro synthesis of wild-type mRNA versus SOD-1 obtained by in vitro synthesis of mutant mRNA.

12. The method of claim 11 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.

13. The method of claim 10 wherein conformer-specific monoclonal or polyclonal antibodies are characterized by differential reactivity shown by binding with mutant in vitro synthesized SOD-1 but not wild-type in vitro synthesized SOD-1, or vice versa.

14. The method of claim 13 wherein conformer-specific monoclonal or polyclonal antibodies are further characterized by a substantial reduction or complete loss of differential reactivity when immunological detection is evaluated using in vitro synthesized SOD-1 that has been denatured prior to antibody binding.

15. The method of claim 9 wherein immunological detection comprises immunoprecipitation of SOD-1 conformers with conformer-specific monoclonal or polyclonal antibodies.

16. A method of determining whether an individual is predisposed to developing ALS, the method comprising:
obtaining a cell free extract derived from cells or tissue taken from an individual, and
identifying at least one SOD-1 conformer in the cell free extract by one or more physical characteristics common to sporadic or familial ALS but distinctive from that of normal individuals.

17. The method of claim 16 wherein said individual has one or more ALS symptoms.

18. The method of claim 16 wherein said individual has a family history of ALS.

19. The method of claim 16 wherein said individual has a mutant SOD-1 protein.

20. The method of claim 16 wherein immunological detection is determined using SOD-1 conformer-specific monoclonal or polyclonal antibodies.
